(19) United States
(12) Patent Application Publication
(10) Pub. No.: US 2017/0049669 A1
(43) Pub. Date: Feb. 23, 2017

(54) A COMPOSITION FOR ORAL APPLICATION AND A METHOD FOR PREPARING THE SAME

(71) Applicant: POSTECH ACADEMY-INDUSTRY FOUNDATION, Pohang-si (KR)

(72) Inventors: Dong Soo HWANG, Pohang-si (KR); Dong Yeop OH, Busan (KR); Jin-Soo AHN, Seoul (KR); Hyung Joon CHA, Pohang-si (KR); Sang-Ho JUN, Seoul (KR); Sung-Won JU, Daegu (KR); Ekavianty PRAJATELISTIA, Pohang-si (KR)

(21) Appl. No.: 15/307,800

(22) PCT Filed: Apr. 29, 2015
(86) PCT No.: PCT/KR2015/004343

§ 371 Date: Oct. 28, 2016

(30) Foreign Application Priority Data
Apr. 29, 2014 (KR) 10-2014-0051815
Apr. 28, 2015 (KR) 10-2015-0059920

(51) Int. Cl. A61K 8/19; A61K 8/29; A61K 8/60; A61K 8/25; A61Q 11/00; A61K 8/24

(52) U.S. Cl. CPC A61K 8/19; A61Q 11/00; A61K 8/24; A61K 8/602; A61K 8/25; A61K 8/29; A61K 2800/412; A61K 2800/413; A61K 2800/92

(57) ABSTRACT

The present invention relates to a composition for oral application and a method for preparing the same, more particularly, to a composition for oral application comprising a complex of specific Lewis base and specific Lewis acid, and a method for preparing the same. The composition for oral application of the present invention has excellent durability, body stability and coatibility, and thus, can effectively treat dentin hypersensitivity, and when applied before dentin hypersensitivity occurs, can prevent dentin hypersensitivity.

### TECHNICAL FIELD

The present invention relates to a composition for oral application and a method for preparing the same, more particularly, to a composition for oral application comprising specific Lewis base and specific Lewis acid, and a method for preparing the same.

This application claims the benefit of Korean Patent Application No. 10-2014-0051815 filed on Apr. 29, 2014 with the Korean Intellectual Property Office, the disclosure of which is herein incorporated by reference in their entirety.

### BACKGROUND OF ARTS

Dental tissues consist of enamel, cement and dentin. The outermost part of the exposed part of tooth is covered with enamel, the outer part of tooth root located in alveolar bone is covered with cement, and at the inner part surrounded with enamel and cement, dentin exists. And, fine tubes named as dentinal tubules are distributed over the dentin.

Dentin hypersensitivity means short and strong pain caused by external stimulations such as thermal, evaporative, tactile, osmotic, chemical stimulations, and the like.

The main cause of dentin hypersensitivity is known to be cervical abrasion (cervical abfracture) due to dental caries, periodontitis, and incorrect toothbrushing, and the like.

Dental caries mainly occurs at the chewing side of tooth or at the proximal surface between teeth and teeth, and they destruct enamel surrounding dentin, and thereby, dentin and dentinal tubules are exposed and dentin hypersensitivity occurs. Such dental caries are mainly generated in the age group of early childhood to adolescence, whose enamel is not sufficiently strengthened.

Periodontitis known as gingival disease occurs due to the damage of periodontal ligament that connects tooth and alveolar bone, wherein simultaneously with the damage of periodontal ligament, cement surrounding tooth root is damaged, and dentin and dentinal tubules of tooth root are exposed, and thus, dentin hypersensitivity occurs. Such a periodontitis appears a lot in the adults after thirty, but most of them are insensible to the disease.

Cervical abrasion (cervical abfracture) mainly occurs due to incorrect toothbrushing, and among the patients complaining of dentin hypersensitivity, cervical abrasion is the most common case. In the cervical portion, the thickness of enamel or cement, which is a mantle surrounding dentin, is very thin, and if one brushes one's tooth from side to side with strong force or enjoys hard foods, enamel or cement at the cervical portion is damaged by external stimulations to expose dentin and dentinal tubules, and thus, dentin hypersensitivity occurs.

Although for the above mentioned dentin hypersensitivity, the mechanism of pain generation and transfer has not been clarified yet, a hydrodynamic theory is widely accepted that enamel or cement surrounding dentin is damaged to expose dentin and dentinal tubules, the flow of dentinal fluid in dentinal tubules is generated, external stimulations are transferred to dental pulp, and thus, pain is felt.

For such a reason, in order to treat the above mentioned dentin hypersensitivity, occlusion of dentinal tubules and desensitization of dental pulp nerves are mainly applied. The desensitization of dental pulp nerves is a treatment of artificially increasing the content of potassium ions so that hypersensitivity of nerves may not be felt, using the fact that neural transmission is controlled by the optimum ratio of sodium ions and potassium ions, and the occlusion of dentinal tubules is a treatment of coating the exposed dentinal tubules with other materials to block exposure to external stimulations, and local fluorine coating, oxalic acid-based material coating, resin coating, and the like are mainly used therefor.

Since among the two methods, the occlusion of dentinal tubules are proceeded a lot in dental clinics, studies on various coating materials having excellent durability, human body stability and coatibility, and the like are required.

### Problem to Solve

It is an object of the invention to provide a composition for oral application having excellent durability, body stability and coatibility.

### Solutions

The present invention provides a composition for oral application comprising one or more Lewis base selected from the group consisting of tannic acid and gallic acid; and one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

The at least a part of the Lewis base and the Lewis acid may exist in the form of a complex wherein Lewis base is coordinated to Lewis acid.

The complex may include one or more selected from the group consisting of a mono-complex, a bis-comlex, and a tris-complex.

And, the composition may comprise the Lewis base and the Lewis acid respectively in the concentration of about 0.01 mg/ml to about 1 mg/ml.

And, the composition may comprise the Lewis base and the Lewis acid at the mole ratio of 0.5:1 to 10:1.

According to one example, the pH of the composition may be in the range of about 3 to about 8.

And, the composition may further comprise silica particles, and the silica particles preferably have a number average particle diameter of 0.05 μm to 5 μm.

The above explained composition may have various dosage forms such as a solution, paste, aerosol or resin and the like.

The present invention also provides a method for preparing a composition for oral application, comprising the step of reacting one or more Lewis base selected from the group consisting of tannic acid and gallic acid; and one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

And, the method may comprise the step of forming a complex of the Lewis base and the Lewis acid.

In the above preparation method, the Lewis base and the Lewis acid may be reacted at the mole ratio of about 0.5:1 to about 10:1.

It is preferable that the step of reacting is carried out at a pH range of about 3 to about 8, and it is preferable that the step of reacting is carried out at a temperature range of 4 to 60° C. and a pressure range of about 0.1 to about 5 atm.

According to one embodiment, the method may further comprise the step of coating silica particles with the complex of the Lewis base and the Lewis acid.

### Advantageous Effect

The composition for oral application of the present invention has excellent durability, body stability and coatibility, and thus, can effectively treat dentin hypersensitivity, and when applied before dentin hypersensitivity occurs, can prevent dentin hypersensitivity.

And, according to the preparation method of the present invention, the composition for oral application may be easily prepared.

### BRIEF DESCRIPTIONS OF DRAWING

FIG. 1 shows the SEM images of various surfaces of teeth specimen with different magnifications.

FIG. 2 shows the SEM images of various cross sections of teeth specimen with different magnifications.

FIG. 3 shows the SEM images of the surface of dental specimen treated with the composition according to one example of the invention with different magnifications.

FIG. 4 shows the SEM images of various cross sections of dental specimen treated with the composition according to one example of the invention with different magnifications.

FIG. 5 shows the SEM images of dentinal tubules on the surface of teeth specimen, before and after treating with the composition according to one example of the invention.

FIG. 6 shows the SEM images of common silica particles and the composition comprising the silica particles according to one example of the invention.

FIG. 7 shows the EDX spectrum of the surface of teeth specimen, after treating with the composition according to one example of the invention.

FIG. 8 shows the EDX spectrum of hydroxyapatite layer collected after treating with the composition according to one example of the invention.

FIG. 9 shows the SEM images of the front and the cross section of teeth specimen, before and after treating with the composition according to one example of the invention.

FIG. 10 shows the SEM images of the front and the cross section of teeth specimen, before and after treating with the composition according to one example of the invention.

FIG. 11 shows the SEM images of the front and the cross section of teeth specimen, before and after treating with the composition according to one example of the invention.

FIG. 12 shows the SEM images of the surface of teeth specimen, after completing toothbrushing of the teeth specimen treated with the composition according to one example of the invention.

### DETAILED DESCRIPTIONS

The composition for oral application comprises one or more Lewis base selected from the group consisting of tannic acid and gallic acid; and one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

And, the method for preparing the composition for oral application of the present invention comprises the step of reacting one or more Lewis base selected from the group consisting of tannic acid and gallic acid; and one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

And, the terms used herein are only to explain illustrative examples, and are not intended to limit the invention. A singular expression includes a plural expression thereof, unless it is expressly stated or obvious from the context that such is not intended. As used herein, the terms “comprise”, “contain” or “have” and the like are intended to designate the existence of practiced characteristic, number, step, constructional element or combinations thereof, and they are not intended to preclude the possibility of existence or addition of one or more other characteristics, numbers, steps, constructional elements or combinations thereof.

And, in case it is stated that each layer or element is formed “on” or “above” each layer or element, it means that each layer or element is formed directly on each layer or element, or that other layers or elements may be additionally formed between the layers or on the object or substrate.

Although various modifications can be made to the present invention and the present invention may have various forms, specific examples will be illustrated and explained in detail below. However, it should be understood that these are not intended to limit the present invention to specific disclosure, and that the present invention includes all the modifications, equivalents or replacements thereof without departing from the spirit and technical scope of the invention.

Hereinafter, the present invention will be explained in detail.

The composition for oral application according to one aspect of the invention comprises one or more Lewis base selected from the group consisting of tannic acid and gallic acid; and one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

According to one embodiment of the invention, at least a part of the Lewis base and the Lewis acid exist in the form of a complex wherein Lewis base is coordinated to Lewis acid.

In general, for treatment of dentin hypersensitivity, a method of occluding exposed dentinal tubules by locally coating fluorine or coating resin on the region where the symptom occurs is used, and recently, studies on a method of promoting regeneration of damaged enamel layers using polydopamine has been progressed.

However, dopamine is very expensive and may be easily oxidized and spoiled according reaction conditions, and it takes a long time to form a coating layer that occludes dentinal tubules, after coating dopamine on the surface of teeth.

Tannin acid is a kind of natural polyphenol synthesized from various plants, and may have the following chemical structure. However, the molecular structure of tannin acid is not limited thereto, and it may exist in the forms of various polymers having various functional groups.

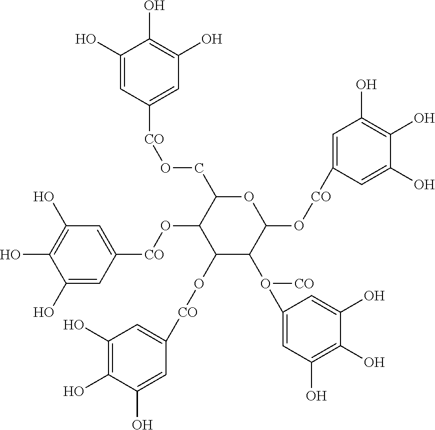

Gallic acid is also a kind of natural biflavonoid antioxidant, and although it may be structurally represented by 3,4,5-trihydroxybenzoic acid, it may exist in various forms of polymers or polyphenols in its natural state.

Since tannin acid and gallic acid have many hydroxyl groups or carboxy groups in the molecules as can be seen from the above explained structure, they may easily bind to macromolecules such as polysaccharides, protein, alkaloid and the like.

Particularly, a galloyl group existing in tannic acid or gallic acid may perform a function as Lewis base, due to lone pairs abundant in the hydroxyl group or carboxy group, and it may form a coordinate bond with various forms of Lewis acid such as ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂), to form a stable complex such as an octahedron structure and the like.

And, since each galloyl group may form a cross link with a center of different Lewis acid, a polymer resin-like strong bond may be formed, thus easily binding on the surface of teeth.

Thus, if the above explained composition comprising Lewis acid and Lewis base is locally applied on the oral cavity region where dentin hypersensitivity occurs, a complex of Lewis base coordinated to Lewis acid that may exist in the composition may form a coating layer on the surface of dentinal tubules exposed outside, and effectively occlude dentinal tubules, thus effectively preventing or alleviating dentin hypersensitivity.

Specifically, when the composition comprising Lewis acid and Lewis base is applied to the oral cavity region where dentin hypersensitivity occurs, that is, to the teeth of which dentinal tubules are exposed, the above explained complex may form a coating layer on the surface of damaged teeth to a thickness of about 10 nm to about 1 μm, preferably about 20 nm to about 500 nm, and thereby, may effectively occlude dentinal tubules with a diameter of about 0.5 to about 2 μm.

After the above explained complex forms a coating layer on the surface of dentinal tubules exposed outside, hydroxyapatite, a main component of teeth, is deposited on the coating layer of the complex, thus forming an hydroxyapatite layer.

The calcium and phosphorous components and the like, which are included in saliva and teeth surface, may be easily deposited on the coating layer of the complex, due to the many functional groups existing in tannic acid or gallic acid molecule, and thus, an hydroxyapatite layer may be easily formed on the coating layer of the complex.

Such a hydroxyapatite layer may completely occlude dentinal tubules that have been exposed, and damaged parts of teeth may be regenerated similarly to the shape of original teeth.

Tannic acid or gallic acid exists in large quantities in natural foods such as a grape, a persimmon, a chestnut, an acorn, green tea and the like, they are polyphenols that have been absorbed into the human body for thousands of years, and the stabilities to the human body are established to some degree.

Since tannic acid or gallic acid may be easily obtained as natural products, and industrially, they may be easily extracted from the bark remaining after processing woods, they have very competitive prices.

And, if the composition is applied on the oral cavity, the above explained complexes of the molecules or small units of tannic acid or gallic acid are initially adsorbed on the surface of teeth, and subsequently, form a coating layer on the surface of teeth, while forming cross linking with other tannic acid or gallic acid molecules, the above explained Lewis acids, or the above explained complex molecules.

Thus, only by applying the composition in the oral cavity in the form of a solution and the like, dentinal tubules may be effectively occluded, thus preventing or alleviating dentin hypersensitivity, and since the formation of the coating layer occurs very rapidly within about 5 to 10 minutes after application in the oral cavity, the composition may be very easily applied for dental treatment and the like.

And, since the cross linking is not easily decomposed under the conditions of oral cavity environment, the composition has very excellent durability, and thus, has very long duration of treatment effect.

The complex of the Lewis base and the Lewis acid may include one or more selected from the group consisting of a mono-complex, a bis-comlex, and a tris-complex.

The mono-complex means a complex wherein two hydroxyl groups of the Lewis base are coordinated to the Lewis acid to form a ligand, the bis-complex means a complex wherein 4 hydroxyl groups of the Lewis base are coordinated to the Lewis acid to form a ligand, and the tris-complex means a complex wherein 6 hydroxyl groups of the Lewis base are coordinated to the Lewis acid to form a ligand.

However, the present invention not necessarily limited thereto, and complexes with various forms and structures may be formed according to the kind of materials used as Lewis acid.

Particularly, if ferrous(II) ion, ferric(III) ion, strontium(III) ion, and/or calcium(II) ion, and the like are used as the Lewis acid, a complex may be formed with these metal ions as a central metal, and if titanium dioxide(TiO₂), ferric oxide(Fe₂O₃), ferrous hydroxide(Fe(OH)₂), calcium oxide(CaO), calcium hydroxide(Ca(OH)₂), silicon(Si), and/or silica(SiO₂), and the like are used as the Lewis acid, the hydroxy groups of the above explained Lewis base may be coordinated to the nanoparticles of these materials to form a complex.

The nanoparticles may have a diameter of about 10 nm to about 1 μm.

Such a complex, for more specific examples, may have the following structural formulae.

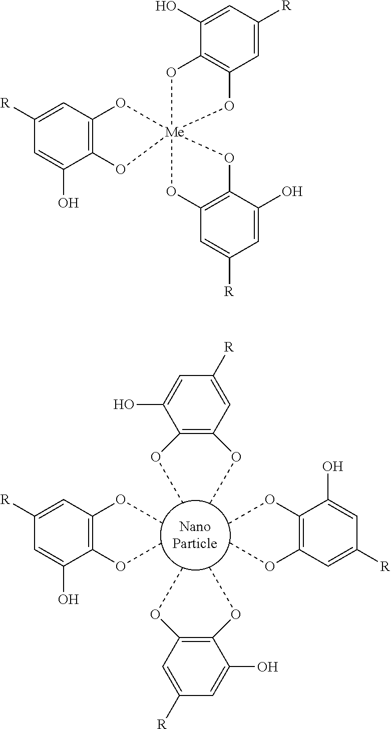

In the above structural formulae, Me means the case wherein ferrous(II) ion, ferric(III) ion, strontium(III) ion, and/or calcium(II) ion, and the like are used as Lewis acid, and metal ions are located in the center of the complex, and Nano Particle means the case wherein nano particles of titanium dioxide(TiO₂), ferric oxide(Fe₂O₃), ferrous hydroxide(Fe(OH)₂), calcium oxide(CaO), calcium hydroxide(Ca(OH)₂), silicon(Si), and/or silica(SiO₂), and the like are used as Lewis acid, and the nano particles of these materials are located in the center of the complex, and the hydroxy groups of the above explained Lewis base are coordinated thereto. However, the complex used in the present invention is not necessarily limited to the above explained structural formulae, and it may consist of complexes having various structures and coordination numbers, according to the kinds of Lewis acid and Lewis base used.

In the above structural formulae, each galloyl group may be in the form of chelate pertaining to the same tannic acid or gallic acid, or they may pertain to different tannic acid or gallic acid molecules and form a cross linking with adjacent complex. And, the complexes may exist together in the state of the composition or in the state of being applied in the oral cavity, according to the external conditions such as a temperature, pH, and the like.

According to one embodiment of the invention, the composition may comprise the Lewis base and the Lewis acid respectively in the concentration of about 0.1 mg/ml to about 1 mg/ml. If the concentrations of the Lewis base and the Lewis acid are lower than the above range, the amount of produced complex is not sufficient, and thus, a coating layer may not be sufficiently formed. However, the present invention is not necessarily limited thereto, and when applied for treatment and prevention of dentin hypersensitivity, the concentrations of the Lewis base and the Lewis acid may vary according to the applied region and the application conditions.

And, it may be preferable that the composition comprises the Lewis base and the Lewis acid at a mole ratio of about 0.5:1 to about 10:1. If the mole ratio of the Lewis base and the Lewis acid is lower than the above range, the amount of the Lewis base may not be sufficient, and it cannot provide many lone pairs to the Lewis acid, and thus, cross linking may not be sufficiently formed, and if the mole ratio of the Lewis base and the Lewis acid is higher than the above range, to the contrary, the amount of the Lewis acid that becomes a center of a complex may be small, and thus, cross linking may not be sufficiently formed.

According to another embodiment of the invention, it may be preferable that the pH of the composition is in the range of about 3 to about 8, more preferably in the range of about 6 to about 7.6. Such a pH range is not only a range having similar acidity to the oral cavity environment, but also a range in which the above explained complex exists in the forms of bis-complex to penta-complex or bis-complex to tris-complex, and may form and maintain strong cross linking. If the pH is lower than the above range, due to high hydrogen ion concentrations, the hydroxyl groups of galloyl groups may not provide a ligand to the central Lewis acid of the complex, and thus, a cross linking may not be effectively formed. And, if the pH is higher than the above range, due to high hydroxide ion concentration, a possibility that the hydroxide ions provide lone pairs to the Lewis acid increases, and thus, a cross linking may not be effectively formed.

For controlling of the pH range, phosphate buffered saline(PBS), tris(hydroxymethyl)aminomethane-HCl, and/or an aqueous sodium hydroxide solution(NaOH) and the like may be used.

According to one embodiment of the invention, the composition may further comprise separate silica particles. The silica particles refer to those separately further included, in addition to the nano silica that is used as the Lewis acid of the composition and forms a complex with tannic acid or gallic acid.

The silica particles may act as a nucleus for forming a coating layer by the complex during the process of forming the above explained cross linking, the surface may be coated with the complex, and when applied for treatment of dentin hypersensitivity, they may perform functions as filler capable of filling damaged parts of teeth. The silica particles of which surfaces are coated with the complex may penetrate into dentinal tubules to occlude dentinal tubules, and the above explained hydroxyapatite layer may be formed thereon, and thus, damaged parts of teeth may be regenerated.

It is preferable that such silica particles have a number average particle diameter of about 0.05 μm to about 5 μm. If the number average particle diameter of silica particles is smaller than the above range, surface coating by the complex may not easily occur, and if it is larger than the above range, silica particles may not penetrate into dentinal tubules. However, the present invention is not necessarily limited thereto.

And, it may be preferable that the silica particles may be included in the amount of about 50 wt % or less based on the total composition.

In addition, even if the above explained composition is applied in the oral cavity, color change of teeth does not seriously occur, and thus, excellent effect may be also obtained in terms of esthetics.

Particularly, when the above explained composition is applied in the oral cavity, ΔE value calculated by the following Equation 1 may be in the range of about 3 to about 25, preferably in the range of about 3 to about 15, more preferably in the range of about 3 to about 6, and thus, only a color change that is difficult to visually identify may be caused, or there may be little color change.

ΔE=\sqrt{ΔL*²+Δa*²+Δb*²} [Equation 1]

And, when the above explained composition is applied in the oral cavity, ΔW* value calculated by the following Equations 2 and 3 may be in the range of about −7 to 20, preferably in the range of about 0 to about 20, more preferably in the range of about 2 to 6, and thus, only a change in tooth whitening index that is difficult to visually identify may be caused, or teeth may rather become bright and tooth whitening effect may be obtained without a separate whitening procedure.

W*=[(a*)²+(b*)²+(L*−100)²]^{1/2} [Equation 2]

ΔW*=W*_{(after)}−W*_{(before)} [Equation 3]

And, the composition may have a dosage form of a solution such as a gargle solution or a coating solution, and the like, paste that can be applied as toothpaste or ointment, and the like, aerosol that can be applied as a spray solution, and the like, or resin that can be used for coating.

In case the composition is prepared in the form of a gargle solution, a patient may repeat a process of directly keeping the gargle solution in one's mouth for a few seconds to a few minutes and then rinsing it, thereby easily treating and preventing dentin hypersensitivity.

In case the composition is prepared in the form of a coating solution or ointment, treatment may be achieved only by dentist's direct application on the affected area, and in case the composition is prepared in the form of resin, treatment may be achieved by coating the resin on the damaged part of teeth in which dentin hypersensitivity occurs and curing.

In case the composition is prepared in the form of toothpaste, the above explained composition may be included as one ingredient of the toothpaste, and treatment and prevention of dentin hypersensitivity may be achieved by repeating toothbrushing using the toothpaste.

However, the present invention is not limited thereto, and the composition may be processed into any dosage forms commonly used in the field to which the present invention pertains, and may be applied for treatment and prevention of dentin hypersensitivity.

Meanwhile, a method for preparing a composition for oral application according to another aspect of the invention comprises the step of reacting one or more Lewis base selected from the group consisting of tannic acid and gallic acid; and one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

At the time of reaction, the reaction methods are not specifically limited, and for example, the reaction may be progressed only by preparing each aqueous solution of Lewis base and Lewis acid, and then, mixing them and stirring.

And, in case metal ions are used as the above explained Lewis acid, various metal salts may be used as a precursor for generating such metal ions.

In the reaction step, it may be preferable that the Lewis base and the Lewis acid are mixed and reacted at a mole ratio of 0.5:1 to 10:1, and the reasons for such a ratio limitation are as explained above.

And, it may be preferable that the reaction step is carried out at a pH range of 3 to 7.5, and the reasons for such a pH limitation are also as explained in the composition.

It may be preferable that the reaction step is carried out at a temperature range of 4 to 60° C. and a pressure range of 0.1 to 5 atm, it is more preferable that the reaction step is carried out at a temperature range of 15 to 40° C. and a pressure range of 0.1 to 2 atm, and it may be most preferable that the reaction step is carried out at a temperature range of 30 to 40° C. and a pressure range of 0.5 to 1.5 atm, which is a similar environment to that of oral cavity, but the present invention is not limited thereto, and can be carried out without limitations of the above explained conditions or devices.

And, the method may further comprise the step of coating silica particles with the complex, which is the product of the above reaction. The coating method is not specifically limited, and for example, surface-coated silica particles may be obtained only by immersing the above silica particles in the composition including each aqueous solution of the Lewis base and the Lewis acid so as to be uniformly dispersed.

According to yet another aspect of the invention, a method for occluding dentinal tubules comprising the step of applying the composition for oral application in the oral cavity region in which dentinal tubules are exposed, to form a coating layer is provided.

And, the method for occluding dentinal tubules may further comprise the step of forming a hydroxyapatite layer on the coating layer, and the details are as explained in the description of the composition for oral application.

Hereinafter, the actions and the effects of the invention will be explained in detail through specific examples of the invention. However, these examples are only presented as illustrations of the invention, and the right scope of the invention is not determined thereby.

### EXAMPLE

### Preparation of Teeth Specimen

A molar teeth without dental caries and restorations, within 3 months after extraction, was prepared. The extracted teeth was stored in a 1% chloramine T solution before testing. The crown of the teeth was removed using a low speed diamond saw, and the teeth was cut to a thickness of 1 mm so that dentinal tubules are exposed, thus preparing a teeth specimen. The cut section was mirror-polished to 3000 mesh using sandpaper, and the specimen was cleaned in a ultrasonic cleaning device sequentially with acetone, ethanol and distilled water, and then, dried at room temperature.

A part of the prepared specimen was taken, and the surface and the cross section were observed by SEM.

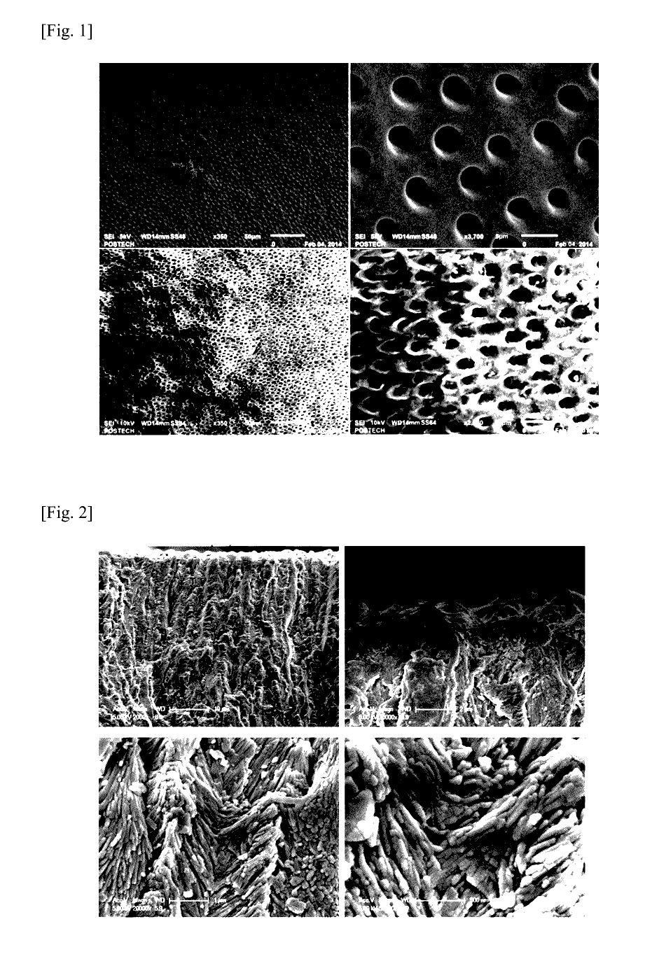

FIG. 1 shows the SEM images of the several parts of the surface of the above prepared teeth specimen with different magnifications.

Referring to FIG. 1, it is confirmed that dentinal tubules are exposed on the surface of the specimen.

FIG. 2 shows the SEM images of various cross sections of the above prepared teeth specimen with different magnifications.

Referring to FIG. 2, it is confirmed that the surface of the teeth specimen is damaged and uneven (right, top)

### Preparation of a Composition for Oral Application

### Example 1

An aqueous solution of tannic acid of a concentration of 0.8 mg/ml and an aqueous solution of ferric chloride (FeCl₃, Sigma Aldrich) of a concentration of 0.2 mg/ml were respectively prepared. Each 5 ml of the aqueous solutions were mixed, and then, a phosphate buffer solution was added dropwise until pH became 7 or more, thus preparing a composition comprising a complex of tannic acid and ferric(III) ion.

### Example 2

To the composition prepared in Example 1, silica particles (Sigma Aldrich) having an average diameter of 50 to 100 nm were added. They were added so that the weight ratio of the composition of Example 1: silica particles became 10:1, and the mixture was stirred so that the silica particles may be uniformly dispersed. Silica particles coated with the tannic acid-iron (III) complex were observed by SEM.

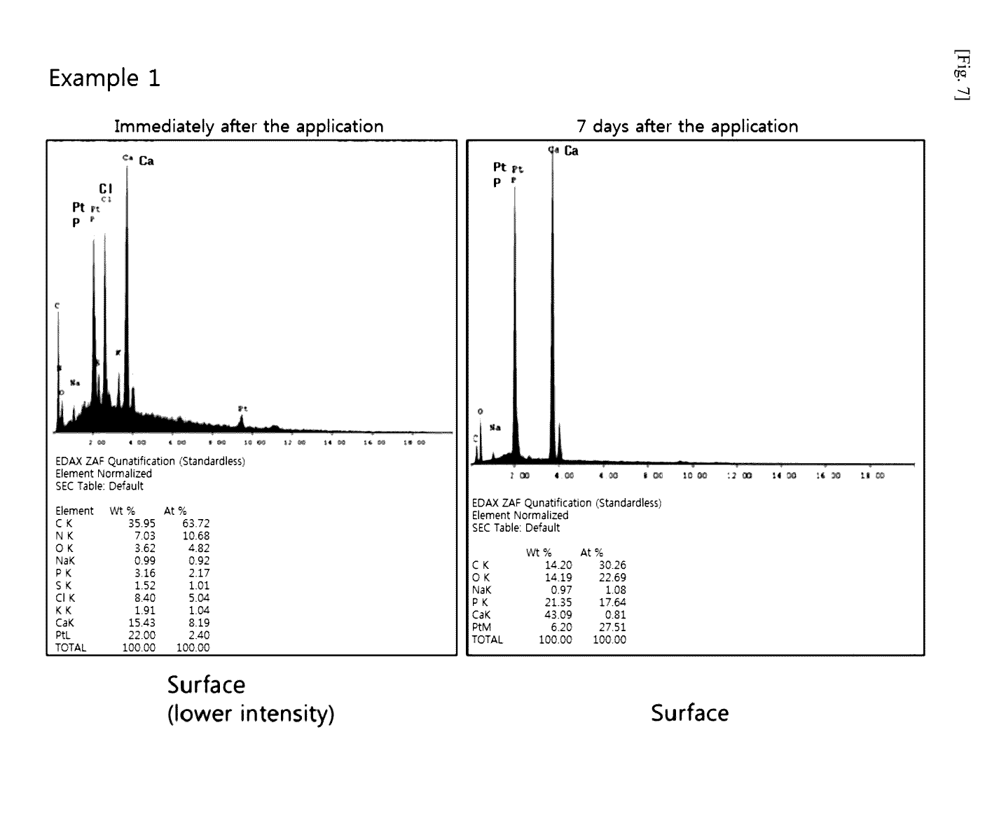

FIG. 6 shows the SEM images of common silica particles and the composition prepared in Example 2.

Referring to FIG. 6, it is confirmed that compared to common silica particles of the left side, the silica particles of the right side has a relatively transparent film on the surface, and it can be interpreted that such as film is formed by the deposition of the tannic acid-iron(III) complex on the surface of the silica particles and the formation of a coating layer.

### Example 3

An aqueous solution of tannic acid of a concentration of 0.8 mg/ml and an aqueous dispersion of titanium dioxide(TiO₂) nanoparticles (particle diameter: 10 nm to 1 μm, manufacturing company: Sigma Aldrich) of a concentration of 0.2 mg/ml were respectively prepared.

Each 5 ml of the aqueous solution and the aqueous dispersion were mixed, and then, a phosphate buffer solution was added dropwise until pH became 7 or more, thus preparing a composition comprising a complex of tannic acid and titanium dioxide nanoparticles.

### Example 4

An aqueous solution of tannic acid of a concentration of 0.8 mg/ml and an aqueous solution of strontium nitrate(Sr(NO₃)₂) of a concentration of 0.2 mg/ml were respectively prepared.

Each 5 ml of the aqueous solutions were mixed, and then, a phosphate buffer solution was added dropwise until pH became 7 or more, thus preparing a composition comprising a complex of tannic acid and strontium ions.

### Example 5

An aqueous solution of tannic acid of a concentration of 0.8 mg/ml and an aqueous dispersion of silica(SiO₂) nanoparticles (particle diameter: 10 nm to 1 μm, manufacturing company: Sigma Aldrich) of a concentration of 0.2 mg/ml were respectively prepared.

Each 5 ml of the aqueous solution and the aqueous dispersion were mixed, and then, a phosphate buffer solution was added dropwise until pH became 7 or more, thus preparing a composition comprising a complex of tannic acid and silica nanoparticles.

### Example 6

An aqueous solution of tannic acid of a concentration of 0.8 mg/ml and an aqueous dispersion of ferric oxide(Fe₂O₃) nanoparticles (particle diameter: 10 nm to 1 μm, manufacturing company: Sigma Aldrich) of a concentration of 0.2 mg/ml were respectively prepared.

Each 5 ml of the aqueous solution and the aqueous dispersion were mixed, and then, a phosphate buffer solution was added dropwise until pH became 7 or more, thus preparing a composition comprising a complex of tannic acid and ferric oxide nanoparticles.

### Example 7

An aqueous solution of tannic acid of a concentration of 0.8 mg/ml and an aqueous dispersion of calcium hydroxide(Ca(OH)₂) nanoparticles (particle diameter: 10 nm to 1 μm, manufacturing company: Sigma Aldrich) of a concentration of 0.2 mg/ml were respectively prepared.

Each 5 ml of the aqueous solution and the aqueous dispersion were mixed, and then, a phosphate buffer solution was added dropwise until pH became 7 or more, thus preparing a composition comprising a complex of tannic acid and calcium hydroxide nanoparticles.

### Example 1-1, and Examples 3-1 to 7-1

The same procedures as Example 1 and Examples 3 to 7 were progressed, except that an aqueous solution of gallic acid was used instead of the aqueous solution of tannic acid.

### Experiment

### Application of the Composition on the Teeth Specimen

The prepared teeth specimen was immersed in the composition of Examples for about 1 minute, and then, it was taken out and washed with distilled water. Immersion and washing were repeated 4 times.

Observation of Teeth Surface

In order to apply an environment similar to that of oral cavity for the teeth specimen to which the composition was applied, artificial salvia was prepared.

To 50 mM of a Tris buffer solution, the salts of calcium chloride(CaCl₂), potassium phosphate(K₂HPO₄), sodium fluoride(NaF) and sodium chloride(NaCl) were added respectively to the following concentrations.

Calcium chloride 2.58 mM, potassium phosphate 1.55 mM, sodium fluoride 1 mg/L, and sodium chloride 180 mM.

0.1M of an aqueous solution of hydrochloric acid was added dropwise thereto so that pH became 7.6, thus preparing artificial salvia.

In the above prepared artificial salvia, the teeth specimen to which the composition was applied was immersed, and it was put in a rotary incubator, which was controlled to 37° C., 100 rpm, and stored for one week.

During the storage period, artificial saliva was newly changed at the cycle of about 24 hours.

After 7 days, the specimen was taken out, washed with distilled water, and a part thereof was taken, and the surface and the cross section were observed by SEM.

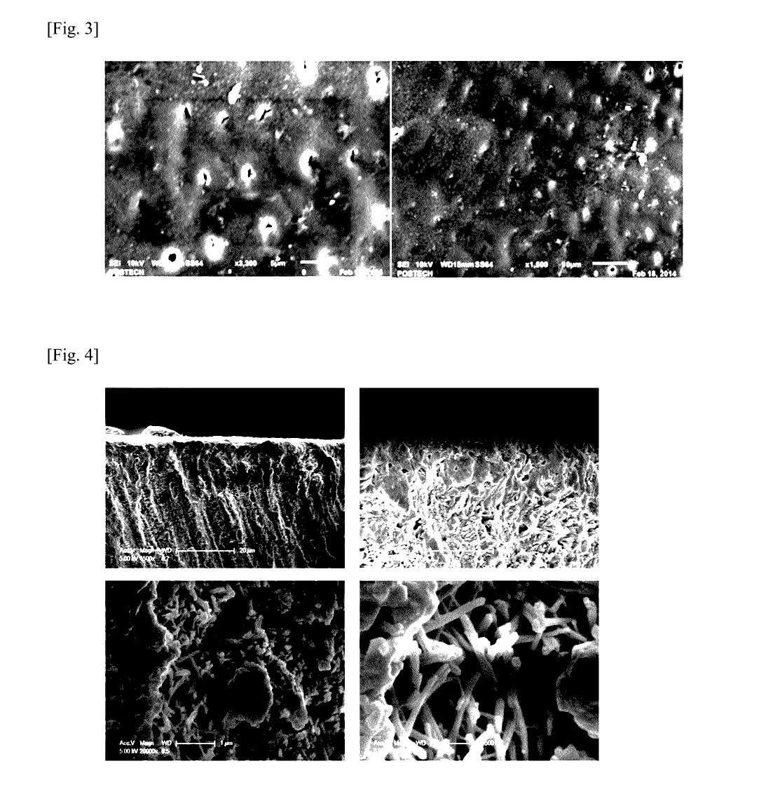

FIG. 3 shows the SME images of the surface of the teeth specimen that was treated with the composition of Example 1.

Referring to FIG. 3, it is confirmed that dentinal tubules are hardly observed on the surface the specimen. Compared to FIG. 1, it is confirmed that dentinal tubules on the surface are almost occluded due to the formation of a coating layer by the composition of the present invention and the formation of a hydroxyapatite layer.

FIG. 4 shows the SME images of various cross sections of the teeth specimen that was treated with the composition of Example 1.

Referring to FIG. 4, it is confirmed that a coating layer is formed on the surface of the specimen, thus forming a uniform height, and particularly, it is confirmed that unevenness due to teeth damage and the like, which was observed in FIG. 2, disappeared (Right, Top). And, referring to the two images of the bottom of FIG. 4, it is confirmed that lump is formed on the surface of teeth, thus confirming that the calcium and the phosphorus components of artificial salvia are deposited on the coating layer of tannic acid-iron(III) complex to form a hydroxyapatite layer.

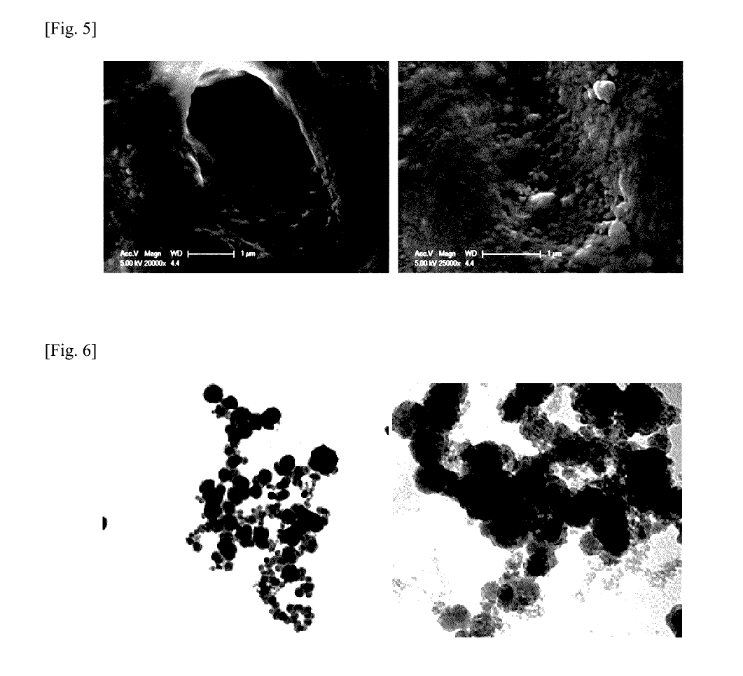

FIG. 5 shows the SEM images of the dentinal tubules of the surface of the teeth specimen, before and after treatment with the composition of Example 1.

Referring to FIG. 5, it is clearly confirmed that dentinal tubules(left) which were observed before treatment with the composition, were occluded due to the treatment of the composition of the present invention.

FIG. 7 shows the EDX spectrum of the surface of the teeth specimen, after treatment with the composition of Example 1.

Referring to FIG. 7, it is confirmed that the contents of calcium and phosphorous largely increased on the surface of the teeth specimen, comparing immediately after application of the composition(left) and 7 days after the application(right). Calcium and phosphorous are the main components of hydroxyapatite, and regarding the large increase in the contents of calcium and phosphorus, it can be interpreted that the calcium and the phosphorus components of artificial salvia were deposited on the coating layer of the tannic acid-iron(III) complex to effectively form a hydroxyapatite layer, and thereby, the damaged parts of teeth were regenerated.

And, after treatment with the compositions of Example 1 and Example 1-1, the generated hydroxyapatite layers were separately collected and the EDX spectra were measured for comparison of the components with common teeth specimen.

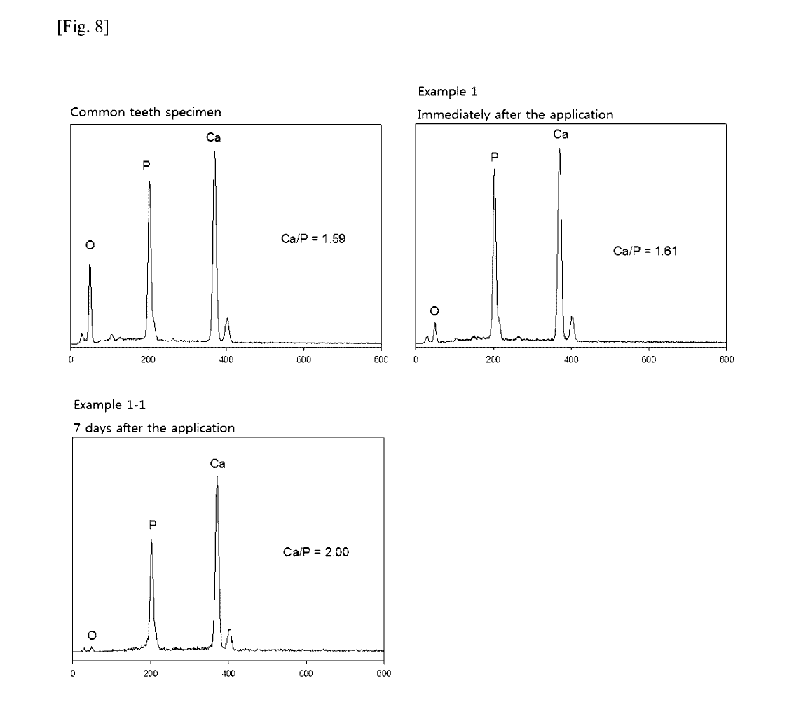

FIG. 8 shows the EDX spectra of the generated hydroxyapatite layers, after treatment with the compositions of Example 1 and Example 1-1.

Referring to FIG. 8, it is confirmed that after treatment with the compositions of Example 1 and Example 1-1, the generated hydroxyapatite has a similar Ca/P ratio to that of common teeth specimen, thus confirming that after treatment with the composition of the present invention, the generated hydroxyapatite layer has a similar composition to common teeth.

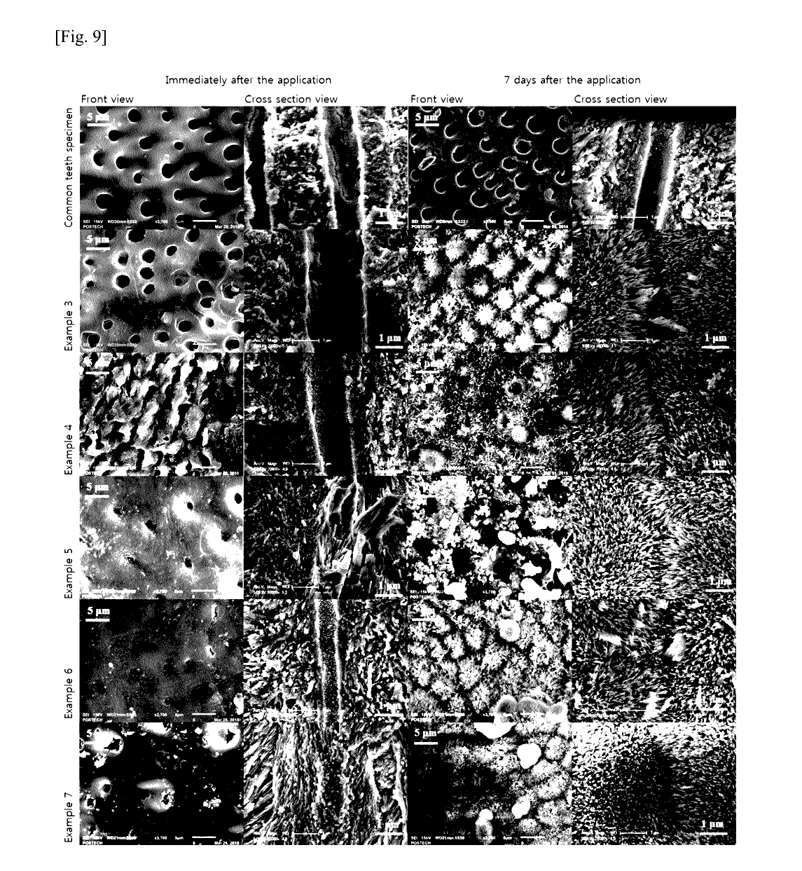

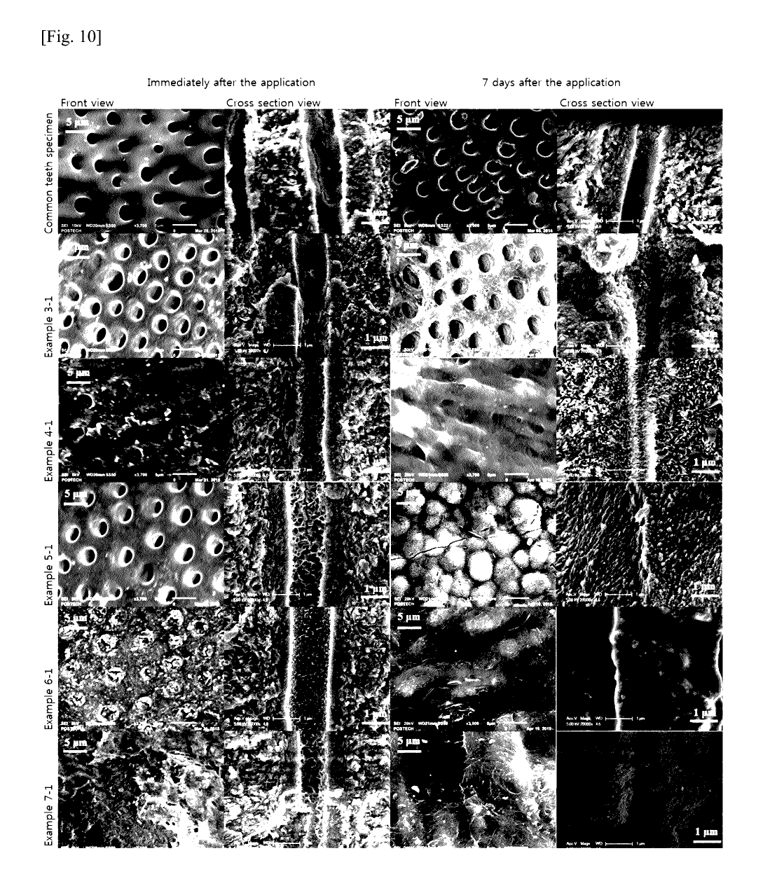

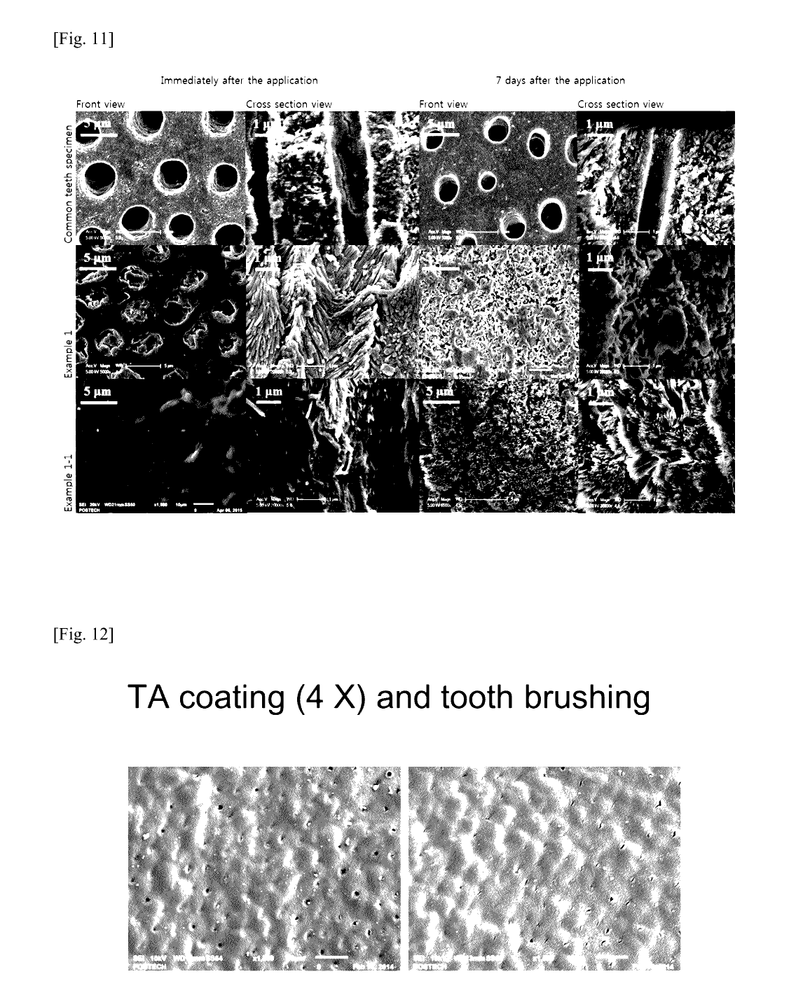

FIG. 9 to FIG. 11 show the SEM images of the front and the cross section of the surface of the teeth specimen, before and after treatment with the compositions of Examples 1, 3 to 7, 1-1 and 3-1 to 7-1.

Referring to FIGS. 9 to 11, comparing the teeth specimen before treatment with the composition and after the application, it is confirmed that dentinal tubules on the surface of teeth were effectively occluded. And particularly, 7 days after the application, lump was formed on the surface, thus confirming that not only the surface of the exposed dentinal tubules but also the inside of the dentinal tubules were occluded, and from this, it can be seen that the calcium and the phosphorus components of artificial salvia were deposited on the coating layer of the above explained complex to form a hydroxyapatite layer.

Durability Confirmation Experiment

A teeth specimen that was treated with the composition of Example 1, in which a hydroxyapatite layer was formed on the coating layer of the tannic acid-iron(III) complex, was prepared in the form of a disc of a diameter of about 10 mm and a thickness of about 1 mm.

For the teeth specimen, toothbrushing was conducted 100 times with the load of 150 g, using an apparatus according to ISO 11609. After the toothbrushing was finished, the surface of the specimen was observed by SEM.

FIG. 12 shows the SEM images of the teeth specimen, after finishing toothbrushing.

Referring to FIG. 12, it is confirmed that dentinal tubules were hardly exposed even after toothbrushing. That is, it is confirmed that the coating layer of the tannic acid-iron(III) complex by the composition of the present invention and the hydroxyapatite layer formed thereon were not eliminated even by toothbrushing under common oral cavity environment conditions, and thus, it is confirmed that the composition of the present invention, when used for treatment of dentin hypersensitivity, is very excellent in terms of durability.

Discoloration Assessment Experiment

For the teeth specimen before and after treatment with the composition for oral application according to Examples 3 to 7, Lab color indexes were measured, and discoloration was assessed by color index change(ΔE) and whitening index change(ΔW*) calculated according to the following Equations 1 to 3, and the results are summarized in the following Table 1.

ΔE=\sqrt{ΔL*²+Δa*²+Δb*²} [Equation 1]

W*=[(a*)²+(b*)²+(L*−100)²]^{1/2} [Equation 2]

ΔW*=W*_{(after)}−W*_{(before)} [Equation 3]

TABLE 1

| | | L | a | b | ΔE | ΔW* |
|---|---|---|---|---|---|---|
| Example 3 | Before | 69.20 | −3.75 | 16.30 | 24.90 | 18.14 |
| | After | 47.15 | −3.62 | 4.74 | | |
| Example 4 | Before | 47.80 | −4.83 | 2.06 | 3.85 | 2.01 |
| | After | 46.00 | −4.69 | 5.46 | | |
| Example 5 | Before | 63.90 | −4.37 | 10.78 | 5.62 | 5.37 |
| | After | 59.12 | −3.96 | 13.70 | | |
| Example 6 | Before | 67.07 | −3.37 | 14.35 | 5.19 | 5.11 |
| | After | 62.39 | −2.90 | 16.55 | | |
| Example 7 | Before | 51.48 | −3.91 | 2.92 | 14.03 | −6.19 |
| | After | 59.96 | −3.30 | 14.08 | | |

Referring to Table 1, it is confirmed that in the case of Examples 3 to 7, even if the compositions are applied on the surface of teeth in oral cavity, color change value is not large.

The ΔE value is in the range of about 3 to about 25 μm all Examples, and thus, it is confirmed that color change is not significant. Particularly, in the case of Examples 4 to 6, the ΔE value is in the range of about 3 to about 6, and it is difficult to visually identify color change of teeth, thus confirming that color change is not significant.

ΔW* value is in the range of about −20 to about 20 μm all Examples, and thus, it is also confirmed that color change is not significant. Particularly, in the case of Examples 3 to 6, the W* value has a positive value, and thus, it is confirmed that teeth whitening effect can be also anticipated by the application of the composition of the present invention.

1. A composition for oral application comprising
one or more Lewis base selected from the group consisting of tannic acid and gallic acid; and
one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

2. The composition for oral application according to claim 1, wherein at least a part of the Lewis base and the Lewis acid exist in the form of a complex of Lewis base coordinated to Lewis acid.

3. The composition for oral application according to claim 2, wherein the complex includes one or more selected from the group consisting of a mono-complex, a bis-comlex, and a tris-complex.

4. The composition for oral application according to claim 1, wherein the composition comprises the Lewis base and the Lewis acid respectively in the concentration of 0.01 mg/ml to 1 mg/ml.

5. The composition for oral application according to claim 1, wherein the composition comprises the Lewis base and the Lewis acid at the mole ratio of 0.5:1 to 10:1.

6. The composition for oral application according to claim 1, wherein the pH is in the range of 3 to 8.

7. The composition for oral application according to claim 1, further comprising separate silica particles.

8. The composition for oral application according to claim 7, wherein the silica particles have a number average particle diameter of 0.05 μm to 5 μm.

9. The composition for oral application according to claim 1, wherein the composition has a dosage form of a solution, paste, aerosol or resin.

10. A method for preparing a composition for oral application, comprising the step of reacting
one or more Lewis base selected from the group consisting of tannic acid and gallic acid; and
one or more Lewis acid selected from the group consisting of ferrous(II) ion, ferric(III) ion, strontium(III) ion, calcium(II) ion, titanium dioxide(TiO₂), ferric oxide(Fe₂O₃), ferrous hydroxide(Fe(OH)₂), calcium oxide(CaO), calcium hydroxide(Ca(OH)₂), silicon(Si), and silica(SiO₂).

11. The method for preparing a composition for oral application according to claim 10, comprising the step of forming a complex of the Lewis base and the Lewis acid.

12. The method for preparing a composition for oral application according to claim 10, wherein the Lewis base and the Lewis acid are reacted at the mole ratio of 0.5:1 to 10:1.

13. The method for preparing a composition for oral application according to claim 10, wherein the step of reacting is carried out at a pH range of 3 to 8.

14. The method for preparing a composition for oral application according to claim 10, wherein the step of reacting is carried out at a temperature range of 4 to 60° C. and a pressure range of 0.1 to 5 atm.

15. The method for preparing a composition for oral application according to claim 10, further comprising the step of coating silica particles with the complex of the Lewis base and the Lewis acid.

16. A method for occluding dentinal tubules, comprising the step of applying the composition for oral application according to claim 1 in the oral cavity in which dentinal tubules are exposed, to form a coating layer.

17. The method for occluding dentinal tubules according to claim 16, comprising the step of forming a hydroxyapatite layer on the coating layer.
